(19) United States
(12) Patent Application Publication
(10) Pub. No.: US 2004/0038320 A1
(43) Pub. Date: Feb. 26, 2004

(54) Methods of detecting cancer using cellular autofluorescence

(76) Inventor: Bhaskar Banerjee, St Louis, MO (US)

Correspondence Address: Harness Dickey & Pierce, Suite 400, 7700 Bonhomme, St Louis, MO, 63105, US

(21) Appl. No.: 10/221,324

(22) PCT Filed: Mar. 9, 2001
(86) PCT No.: PCT/US01/07589

(51) Int. Cl. G01N 33/574; G01N 1/30; G01N 33/48

(52) U.S. Cl. 435/7.23; 435/40.5

(57) ABSTRACT

Methods useful in detecting abnormal cells and tissues based upon measurement of tryptophan-associated autofluorescence are disclosed. The methods involve exposing cells to ultraviolet light; measuring autofluorescence at a wavelength indicative of tryptophan emission; and determining that the cells constitute abnormal tissue of emission intensity exceeds that of normal cells. The methods can detect and distinguish abnormal cell development in metaplasia, hyperplasia, dysplasia and cancer. The method also detects the presence of inflammation.

### CROSS REFERENCE TO RELATED APPLICATION

This application is a continuation-in-part application of prior application Ser. No. 09/522,557, filed Mar. 10, 2000 which is incorporated herein by reference.

### BACKGROUND OF THE INVENTION

(1) Field of the Invention

This invention relates generally to detecting abnormal cell development and more particularly, to detecting cells which are neoplastic or metaplastic by measuring cellular autofluorescence.

(2) Description of the Related Art

The development of cancer cells can involve a progressive abnormal cell development which ultimately becomes a malignant neoplasm. One example of such progression is believed to occur in the development of esophageal adenocarcinoma. This disease progression may be initiated by the reflux of acid and bile into the esophagus which produces an inflammation and cell damage. A series of events termed the metaplasia-dysplasia-carcinoma sequence can result. The normal squamous cell epithelium of the esophagus becomes replaced by columnar epithelium in what is termed Barrett's disease. Subsequently, the cells become dysplastic and this ultimately progresses to adenocarcinoma. For review see Tselepis et al., Digestion 61:1-5, 2000.

The progressive nature in the development of cancer can allow early detection of the disease which can be crucial inasmuch as the survival rate for cancer patients increases with early detection. Numerous invasive and non-invasive approaches have been used in the detection of cancer including tissue biopsy, x-ray screening, computerized tomography, and the use of molecular markers. One non-invasive approach in attempts to detect cancerous tissue has involved the use of autofluorescence, i.e. the measurement of fluorescence emission upon exposing a target region to ultraviolet light. Autofluorescence occurs when fluorophores in the target are excited by light of one wavelength and subsequently emit light at a longer wavelength. Human and animal tissues exhibit an autofluorescence from fluorophores which occur naturally in the tissues. A number of endogenous fluorophores have been studied of which tryptophan, collagen type IV and NADH have been reported as possible sources responsible for three emission maxima seen in normal, adenomatous and malignant tissues (Banerjee et al., Am. J. Med. Sci. 316:220-226, 1998). In studying the emission spectrum of these tissues, it was reported that the intensity of one emission peak increased progressively with dysplasia and carcinoma and that tryptophan is a likely cause of the emission peak. Id. Nevertheless, this earlier work was intended to determine whether all the emission peaks were present in tissues of different histologic grades and whether the emission peaks showed any shift associated with neoplasia and measurement of fluorescence intensity was not the object of the study. Thus, this earlier work provided no suggestion that the observation of increased emission intensity with disease progression might be applicable to an approach for detecting neoplasia or the degree of progression of the neoplasia.

Cancer detection methods based upon measuring autofluorescence have the advantage of avoiding the introduction of exogenous agents such as radionuclides, monoclonal antibodies, or exogenous fluorophores. The use of exogenous agents can add to the cost of diagnostic methods using such agents and the time required to administer such agents and allow them to become incorporated into the examined tissue can increase the duration and complexity of the test. Exogenous agents also can introduce the risk of adverse reaction.

Earlier work using autofluorescence to detect cancer has involved measurement of autofluorescence from whole tissue. Such approaches, however, predominantly measure non-specific autofluorescence which is emitted from a variety of extracellular components of whole tissue. Extracellular components which exhibit autofluorescence include collagen and elastin as well as non-tissue elements such as blood and blood vessels. While these extracellular components may change during the progression from normal to cancerous tissue, the changes do not constitute changes in the cancer itself and as a result the changes in extracellular autofluorescence are not specific indicators of the presence of cancer cells. For example, the presence of inflammation can lead to misclassification of cancer cells due to the presence of inflammatory cells. (Ramanujam et al., Photochem. Photobiol. 64:720-735, 1996). Thus, the use of autofluorescence to detect cancerous tissue has not, heretofore, been able to distinguish between specific cellular changes indicative of cancer and non-specific extracellular changes.

### SUMMARY OF THE INVENTION

Accordingly the inventor herein has succeeded in discovering that cell-specific autofluorescence can be measured by measuring tryptophan-associated autofluorescence and that changes in cell-specific autofluorescence from that in normal cells provides a simple and predictive approach for detecting abnormal cells in the progressive development of neoplasia.

Thus, in one embodiment, the present invention involves a method of detecting neoplasia. The method comprises exposing cells suspected of constituting neoplastic to ultraviolet light and measuring autofluorescence. The autofluorescence measured comprises a wavelength indicative of a tryptophan spectrum and which lies within a range of wavelengths of from about 300 to about 400 nm. The cells are determined to be neoplastic if emission intensity is higher than emission intensity of cells which do not constitute neoplasia. Cells which show a ratio higher than about 1.3 for emission intensity to emission intensity of cells which do not constitute neoplasia are deemed to constitute neoplasia. The measured cellular autofluorescence increases with increasing degree of neoplasia such that the degree of neoplasia can be determined by assessing the amount of autofluorescence. Thus the magnitude of cellular autofluorescence increases with the degree of severity of the neoplasia, i.e. the greater the progression of the neoplasia from hyperplasia to dysplasia to cancer.

The cells constituting neoplasia can constitute hyperplasia, dysplasia or cancer. Cells showing a ratio of from about 1.3 to about 1.7 for autofluorescence emission to that of cells not constituting neoplasia are deemed to constitute hyperplastia; cells showing a ratio of from about 1.7 to about 2.5 are deemed to constitute dysplasia and cells showing a ratio higher than about 2.5 are deemed to constitute cancer.

In one aspect of this embodiment, the autofluorescence can be measured over a bandwidth which lies within a range of from about 300 to about 400 nm, preferably a bandwidth of about 100 nm, more preferably, a bandwidth of about 50 nm, more preferably, a bandwidth of about 30 nm, more preferably, a bandwidth of about 20 nm and most preferably, a bandwidth of about 10 nm or less. It is preferred that the bandwidth include the peak of the tryptophan spectrum of about 330 nm.

In a further embodiment, the present invention is directed to another method of detecting neoplastic cells. The method comprises exposing cells suspected of being neoplastic to ultraviolet light; measuring autofluorescence emission from the cells over a bandwidth other than a bandwidth of 20 nm bandwidth wherein the bandwidth lies within a range of from about 300 to about 400 nm; and determining that the cells constitute neoplasia if emission intensity is higher than emission intensity of cells which do not constitute neoplasia. Preferably, the cells are determined to constitute neoplasia if the ratio of emission intensity to that of cells which do not constitute neoplasia is higher than about 1.3.

The degree of neoplasia can be determined by assessing the magnitude of autofluorescence inasmuch as the magnitude of cellular autofluorescence increases with the degree of severity of the neoplasia. Cells showing a ratio of from about 1.3 to about 1.7 for autofluorescence emission to that of cells not constituting neoplasia are deemed to constitute hyperplastia; cells showing a ratio of from about 1.7 to about 2.5 are deemed to constitute dysplasia and cells showing a ratio higher than about 2.5 are deemed to constitute cancer.

The present invention, in another embodiment, is directed to a method of detecting metaplasia. The method comprises exposing cells suspected of constituting metaplasia to ultraviolet light and measuring autofluorescence emission from the cells at a wavelength indicative of a tryptophan emission spectrum. The cells are determined to constitute metaplasia if emission intensity differs from that of cells which do not constitute metaplasia. In one aspect of this embodiment, the method is directed to a method of detecting esophageal metaplasia of squamous cells to columnar cells. Most of such esophageal metaplasias are diagnosed as Barrett's disease. The esophageal metaplastic change from squamous cells to columnar cells has been discovered to produce a decrease in cellular autofluorescence. Thus, cells are determined to constitute the esophageal metaplasia if emission intensity is less than emission intensity of cells which are not metaplastic. Preferably, the cells are determined to constitute esophageal metaplasia if the ratio of emission intensity to that of cells which do not constitute metaplasia is less than about 0.65.

Autofluorescence emission can be measured at a wavelength which lies within about 300 to about 400 nm, preferably, at 330 nm. Autofluorescence emission can also be measured over a bandwidth which lies within a range of from about 300 to about 400 nm, the bandwidth is preferably about 100 nm, more preferably about 50 nm, still more preferably, about 30 nm and most preferably, about 10 nm or less. The bandwidth, preferably, includes the peak of the tryptophan spectrum of about 330 nm.

The present invention, in yet another embodiment, is directed to a method of detecting inflammation. The method comprises exposing cells suspected of constituting inflammation to ultraviolet light and measuring autofluorescence emission from the cells at a wavelength indicative of a tryptophan emission spectrum. The cells are determined to constitute inflammation if the ratio of emission intensity to that of cells which do not constitute inflammation is less than about 0.75. The method can also distinguish inflammation from metaplasia. Thus, in one aspect of this embodiment, the cells are determined to constitute inflammation if the ratio of emission intensity to that of cells which do not constitute inflammation is less than about 0.75 and greater than about 0.65.

Autofluorescence emission can be measured at a wavelength which lies within about 300 to about 400 nm, preferably, at 330 nm. Autofluorescence emission can also be measured over a bandwidth which lies within a range of from about 300 to about 400 nm, the bandwidth is preferably about 100 nm, more preferably about 50 nm, still more preferably, about 30 nm and most preferably, about 10 nm or less. The bandwidth, preferably, includes the peak of the tryptophan spectrum of about 330 nm.

Another embodiment of the present invention is directed to a method of detecting neoplasias in the presence of inflammation. The method comprises exposing a region suspected of containing cells constituting neoplasia in the presence of inflammation to ultraviolet light and measuring autofluorescence at a wavelength indicative of a tryptophan emission spectrum. The region is determined to contain cells constituting neopolasia if emission intensity is higher than emission intensity of cells which do not constitute neoplasia.

Cellular autofluorescence increases with the degree of severity of the neoplasia. Cells showing a ratio of from about 1.3 to about 1.7 for autofluorescence emission to that of cells not constituting neoplasia are deemed to constitute hyperplastia; cells showing a ratio of from about 1.7 to about 2.5 are deemed to constitute dysplasia and cells showing a ratio higher than about 2.5 are deemed to constitute cancer.

Autofluorescence emission can be measured at a wavelength which lies within about 300 to about 400 nm, preferably, at 330 nm. Autofluorescence emission can also be measured over a bandwidth which lies within a range of from about 300 to about 400 nm, wherein the bandwidth is preferably about 100 nm, more preferably about 50 nm, more preferably, about 30 nm, more preferably, about 20 nm and, most preferably, about 10 nm or less. The bandwidth, preferably, includes the peak of the tryptophan spectrum of about 330 nm.

In the methods of this invention, it is preferred that the emission intensity be compared to that of control cells which do not constitute neoplasia by calculating the ratio of emission intensities, however, any method available to the skilled artisan can be used to compare autofluorescent emission of cells being tested to that of control cells. The cells, which can be in vivo or in vitro, are exposed to a wavelength capable of eliciting an emission at a wavelength or wavelenths indicative of tryptophan fluorescence emission. In embodiments in which the cells are in vivo, the ultraviolet light can be delivered by any of a number of approaches including one approach comprises delivering ultraviolet light delivered directly or by using a lens or a mirror or through a fiber optic bundle inserted through a biopsy channel of an endoscope or laparoscope or through a needle. In embodiments in which the cells are in vitro, the cells can be obtained by any method including from a cell washing or a cell brushing or from a tissue biopsy.

The cells suspected of constituting neoplasia can be from a gastrointestinal organ, lung, bladder, ureter, cervix, skin, bile duct, pancreatic duct, liver, kidney, uterus, ovaries, fallopian tubes, mouth, throat or nasopharynx. Cells from a gastrointestinal organ are typically from the esophagus or colon. The cells are, preferably, but not necessarily, from the same organ type as the cells which are not metaplastic. The cells can be living cells or fixed, such as with formalin.

The present invention is also directed to apparatus configured to detect neoplastic cells in accordance with the methods of the invention.

Among the several advantages achieved by the present invention, therefore, may be noted the provision of methods and apparatus for detecting neoplastic cells using cellular autofluorescence which facilitates the early detection of cancerous cells; the provision of methods for distinguishing cancerous changes in tissue from extracellular changes which are non-specific to cancer; the provision of methods for the early detection of cancer which is simple to practice and avoids the need for complex, subjective pathological comparisons of tissues; the provision of methods for detecting metaplasia; the provision of methods for detecting inflammation; and the provision of methods for the early detection of cancer which exhibits a reliability which is unaffected by tissue inflammation.

### BRIEF DESCRIPTION OF THE DRAWINGS

FIG. 1 illustrates an exemplary autofluorescence emission spectrum of a tissue sample.

FIG. 2 illustrates an emission spectrum of cultured cells.

FIG. 3 illustrates an emission spectrum of tryptophan in aqueous solution.

FIG. 4 illustrates emission spectra of normal colon cells and cancerous colon cells.

FIG. 5 illustrates emission spectra of membranous and cytosolic fractions derived from cells obtained from normal colonic tissue.

FIG. 6 illustrates autofluorescence ratios of a tryptophan-associated emission peak for different types of esophageal tissue.

FIG. 7 illustrates autofluorescence ratios of the tryptophan-associated peak for different types of colonic tissue studied.

### DETAILED DESCRIPTION OF THE INVENTION

The present invention is directed to methods and apparatus for detecting neoplasia in vitro and in vivo using cellular autofluorescence. By neoplasia or neoplastic tissue or cells which constitute neoplasia, it is meant that the cells or tissue are in an abnormal state of development. Neoplasia, as used herein, is intended to include hyperplasia, dysplasia and cancer.

Hyperplasia is an abnormal increase in the number of cells in a tissue although the cells themselves are generally considered to be normal. Hyperplasia can be initiated by any of a number of stimuli. Dysplasia is a neoplastic proliferation of abnormal cells which can also be pre-cancerous. Examples of dysplasias are adenomas and squamous cell dysplasias. Cancer cells are cells present in a malignant tumor characterized by the properties of diminishment or absence of control over replication, invasiveness and ability to metastasize.

The present invention also encompasses the detection of metaplasia and cells which constitute metaplasia. By metaplasia or cells which constitute metaplastia, reference is made to the replacement of one type of adult, differentiated cell type with another adult cell type, often in an adaptive response to chronic injury. The replacement cell type is usually not present in the tissue in which it is found. The abnormal cell types can reflect the progressive development of cancer cells in a sequence that advances from metaplasia to dysplasia and ultimately to cancer cells as is seen in the development of esophageal adenocarcinoma.

The methods of the present invention are based upon measurement of the progressive changes in cellular autofluorescence. The amino acid, tryptophan is commonly found in cells where it exhibits a strong fluorescence. Cellular autofluorescence due to the endogenous fluorophore, tryptophan has been found to be a measurable indicator of the presence and the degree of cellular changes involved in the progression to cancer. Although there are many extracellular sources of autofluorescence, including collagen, elastin, blood vessels and leukocytes, tryptophan is primarily a cellular constituent and measurement of the level of cellular autofluoresence due to tryptophan provides a sensitive, specific and reproducible assessment of the presence of cells in various stages in the progressive changes in the development of cancer.

The term tissue as used herein refers to both in vitro and in vivo tissues including tissues or organs in a live human or animal as well as samples of cells obtained from a human or animal, such as in cytology such as in the examination of a film of cells on a glass slide. Such tissues constitute biological samples which can be present in or obtained from a human or animal patient or subject.

The methods and apparatus of the present invention can be used in connection with the detection of early cancer, or pre-cancer, or dysplasia.

The methods of the present invention involve exposing cells to ultraviolet light in order to obtain an autofluorescence from the cells. The ultraviolet light source can be any ultraviolet light source capable of providing an excitation of the cellular fluorophore, tryptophan. Suitable ultraviolet sources include xenon arc lamps, mercury vapor lamps, metal halide lamps, ultraviolet lasers and the like.

Ultraviolet light suitable for excitation and eliciting of tryptophan emission wavelengths is typically at wavelengths below the expected emission wavelengths. The spectrum of tryptophan emission is over a range of from about 300 nm to about 400 nm, with the peak emission about 330 nm. Thus, excitation wavelengths include wavelengths which are preferably 400 nm or less, more preferably 330 nm and less, more preferably, 300 nm or less and most preferably, 290 nm or less. As used herein, the term “about” is intended to encompass a range of values which are 10% below and 10% above a stated value so that, for example, about 300 nm is intended to encompass 270 nm to 330 nm.

It is also possible to elicit autofluorescence using multi-photon excitation such as for example, with a pulsed laser in which cells are exposed to two photons at double any wavelength within the range of wavelengths which will elicit a tryptophan fluorescence emission or to three photons at three times a tryptophan excitation wavelength and the like. Thus, instead of 300 nm excitation, one can use 600 nm with two photons or 900 nm with three photons and instead of 290 nm, one can use 580 nm with two photons or 870 nm with three photons.

The use of autofluorescence emission measurements to determine that cells are abnormal or from an abnormal tissue is preferably based upon a calculation of the ratio of emission to that of control cells. Nevertheless, any approach available to the skilled artisan can be used to compare emission of the test cells with that of control cells such as comparison of absolute values of emission which are typically measured in arbitrary units, comparison of areas under the curves for a spectral region corresponding to tryptophan emission, comparison of values for a single wavelength or for a bandwidth indicative of a tryptophan emission and the like.

For measurements in which a wavelength indicative of a tryptophan curve is used, the measured wavelength, preferably lies within a range of from about 300 nm to about 400 nm. Preferably, the wavelength is near the peak of tryptophan emission, i.e. about 330 nm. For measurements in which a bandwidth is assessed, the bandwith is a bandwidth indicative of a tryptophan emission spectrum which, preferably, lies within a range of from about 300 nm to about 400 nm. Preferably, the bandwidth encompasses, or it is at least near the peak of, tryptophan emission, which is about 330 nm. The bandwidth is preferably a bandwidth of about 100 nm, more preferably, about 50 nm, more preferably, about 30 nm, more preferably, about 20 nm and most preferably, about 10 nm or less.

Autofluorescence emissions can be measured by any suitable fluorescence detector such as, for example, a photodiode. Fluorescence emission at a wavelength or from a bandwidth within the spectral range of tryptophan fluorescence emission can be distinguished by using appropriate filters which are well known in the art. For example bandpass filters can be used which transmit a well-defined band of ultraviolet light and reject unwanted light at wavelengths above and below the selected bandpass such as Fabry-Perot type bandpass filters which are commercially available.

Moreover, commercially available spectrofluorometers can be used to perform the methods of the present invention in which excitation and emission wavelengths can be selected.

The methods of the present invention determine that cells or tissues are abnormal based upon a difference in autofluorescence detected from the tissue compared to that of normal tissue. The magnitude of any change compared to control as well as whether the change is an increase or decrease compared to control are both relevant to the determination. Thus it has been discovered that an increase in autofluoresence intensity of at least about 30% to about 50% above normal, i.e. a ratio of about 1.3 to about 1.5 that of normal cells occurs with hyperplasia.

For the purposes of simplifying the method, a value can be selected as a cut-off point and cells showing values above the cut-off point are deemed to be abnormal whereas cells showing values below the cut-off point are deemed to constitute normal or less diseased cells. The selection of such cut-off values involves balancing the degree to which the method will detect false negatives and false positives. False negatives are cells which are concluded to be normal while, in fact, the cells are abnormal and false positives are cells which are concluded to be abnormal while, in fact, the cells are normal. Thus, a cut-off value which is too low will tend to detect more false positives and a cut-off value which is too high will tend to detect more false negatives.

The preferred cut-off value for concluding that cells or tissue constitute hyperplasia is a value of about 30% above normal, i.e. a ratio of 1.3, and, less preferably, a value of about 40% above normal, i.e. a ratio of 1.4.

Cells or tissue which constitutes dysplasia show an increase in autofluorescence intensity of at least about 60% to about 100% above normal, i.e. a ratio of about 1.6 to about 2.0. For the purposes of simplifying the method, a value of about 70% above normal, i.e. a ratio of about 1.7, and, less preferably, a value of about 60% above normal, i.e. a ratio of about 1.6, or a value of about 80% above normal, i.e. a ratio of about 1.8, can be used as a cut-off point in concluding that cells or tissue constitute dysplasia.

Cells or tissues can be deemed to constitute cancer in accordance with the above simplification of the methods of this invention, if the cells or tissue show an increase in autofluorescence at a cut-off point of, preferably, at least about 150% above normal., i.e. a ratio of at least about 2.5 or greater, and, less preferably, at least about 200% above normal, i.e. a ratio of at least about 3.0 or greater.

It is to be understood that the values given above are intended to reflect minimal changes indicative of the abnormal cells or tissues and cells or tissues constituting hyperplasia, dysplasia or cancer can show higher values than those indicated above.

Decreases in autofluorescence also occurs with conditions reflecting abnormal changes in cells. Thus, it has been discovered that inflammation results in a decrease in autofluorescence intensity of about 25% or more, i.e a ratio of 0.75 or less. Esophageal metaplasia involving a change from squamous cells to columnar cells also results in a decrease in autofluorescence intensity and this change amounts to a decrease in autofluorescence intensity of about 35% or more, i.e. a ratio of 0.65 or less.

The method of the present invention can also be used to detect metaplasias other than the metaplastic change of squamous cells to columnar cells as is seen in esophageal metaplasia. The change in autofluorescence for a given type of metaplasia with the change in cell type can be readily ascertained by the skilled artisan and the subsequent determination whether the metaplasia is present in a given sample of test cells is based upon such earlier determination.

Most cancers first develop in the epithelial layer. As cancers progress and enlarge, they grow into deeper tissue layers and reach nearby blood or lymph vessels to metastasize throughout the body. Spread can also occur directly to adjacent tissues and organs. The key to early diagnosis of cancer is to detect malignant or dysplastic growths before they metastasize, when they are still localized to the epithelial lining of organs. In practice, this will allow neoplasia to be detected in high risk patients before they can be seen by the naked eye. Early neoplasia can then be treated and potentially cured.

When the epithelial surface of a tissue such as the intestine is illuminated, and an assessment of the tryptophan autofluorescence made, a measure of cellular fluorescence is achieved, with very little fluorescence from extracellular molecules.

At the low wavelengths used to generate this fluorescence (at about 280 nm to 310 nm), substantial penetration to deeper tissue layers does not take place. Both these factors combine to give a measure of autofluorescence that is largely from epithelial cells.

This technique would also enable malignant cells with a higher tryptophan content in areas deeper to the mucosa to be detected, if the optical process is taken there by way of a needle or trocar or other instrument, or if a sample of tissue from such a source were to be assessed after it is removed. Mutli-photon excitation is another way of reaching the deeper layers, as well as using confocal (microscopy) techniques.

During carcinogenesis, cellular replication increases without control; synthesis of cellular molecules including proteins is accelerated as each cell prepares to divide. Microscopically, cells are seen to be denser, with large dense nuclei and nucleoli taking up progressively larger portions of the cellular space, resulting in higher nucleus to cytoplasm ratio. These are among the criteria used by pathologists when assessing tissues for the presence and degree of neoplastic change. Measurement of tryptophan autofluorescence enables a comparable determination of these factors to be made in real time.

The density of cells in a given volume of tissue increases with progression of neoplasia, whilst the extra-cellular molecules take up a proportionately smaller space. Autofluorescence from extra cellular molecules such as collagen and elastin will be expected to simultaneously decrease.

Inflammation is a very common condition in living tissues, caused by a number of processes including infection, trauma, burn, chemical injury, toxins, drugs and disorders of the immune system. Inflammation can be acute or chronic, it can exist by itself, be present alongside dysplasia or cancer and even lead to the development of cancer. The presence of inflammation can interfere with the microscopic process of detecting dysplasia, as in Barrett's esophagus with dysplasia and esophagitis (active inflammation of the esophagus). It is therefore most important for any optical diagnostic method, not just autofluorescence, to be able to account for tissue inflammation. If a technique cannot distinguish between inflammatory and neoplastic changes, its use in cancer detection will be limited. If neoplasia and inflammation cause similar changes in autofluorescence, it will be very difficult to detect malignancy in the presence of inflammation, due to false positive results.

Inflammation is a complex process. Tissue inflammation results in vascular engorgement, changes in microvascular blood flow, exudation of protein rich fluid and migration of leukocytes. Endogenous chemical mediators of inflammation in the inflamed area further increase vascular permeability and the inflammatory response. Circulating leukocytes including neutrophils, monocytes, basophils and bosinophils arrive at the tissue site. Humoral mediators including the complement and kinin systems, histamine, interleukin 1, tumor necrosis factor and others are found in inflamed tissue. T and B lymphocytes interact among themselves and also with monocytes, macrophages, immunoglobulins and the complement system. There may also be different grades of epithelial injury. In chronic inflammation, lymphocytes may predominate the cellular reaction. In some cases, macrophages fuse to form giant cells and granulomas. Fibrosis of the inflamed site can lead to scar tissue formation and a distorted architecture. Inflammation is therefore a complex of cellular, humoral and hemodynamic changes that evolve with time, the nature of the causative agent, host response and therapy.

The complex changes in inflammation have to be accounted for in any process that involves optical detection. Inflamed tissue becomes edematous which affects optical properties of tissues. The overall effect of inflammation on tissue autofluorescence due to tryptophan would be a slight decrease, probably due to the collection of excess fluid in inflamed tissue that will reduce cellular density and hence the tryptophan associated autofluorescence.

An epithelium is the cellular covering of both internal and external body surfaces, including the lining of vessels and cavities. Epithelia are classified by the shape of the cells and the number of layers (stratified or pseudo-stratified). Epithelial cells can be squamous (saucer shaped), columnar, transitional, cuboidal, ciliated (bearing cilia), pyramidal, rod shaped, etc. The differences in the structure or shape of these cells will have different effects on the optical or other physical properties of any electromagnetic radiation (including light) that interacts with them. Such differences will enable one to detect and hence identify different cell types without the use of a microscope or the naked eye. Optical and other physical techniques can thus be used to detect epithelial changes in an automated fashion, once the changes associated with a particular cell type or epithelium is demonstrated. This will apply to autofluorescence and other techniques including but not limited to: time resolved fluorescence, excitation scans, elastic scattering, Raman spectroscopy and opto-acoustic methods. Detection of a cell type that is not normally present in an organ often indicates the presence of a disease. An example is Barrett's esophagus, in which the squamous lining of the esophagus is replaced by columnar cells (specialized intestinal metaplasia).

The detection methods of the present invention are also applicable with fixed tissue samples. As illustrated below, cells fixed in 10% formalin solution still show autofluorescence which can be used in distinguishing abnormal cells and tissues from normal cells and tissues. It is believed that the detection methods of the present invention are applicable to tissues fixed by any method which maintains cell structure and chemical composition including the use chemical fixing agents such as formalin, formaldehyde, acetic acid, acetone, chromium trioxide, ethanol, glutaraldehyde, mercuric chloride, methanol, osmium tetroxide, picric acid, potassium dichromate, trichloracetic acid and the like as well as fixing by freeze drying, with microwaves and the like.

The methods of the present invention are based upon application of cell-specific autofluorescence in the tissue diagnosis of malignancy. When whole tissue is excited at a wavelength in the range of about 230 nm to about 350 nm, for example at about 290 nm, the autofluorescence emitted from the tissue and measured at an emission wavelength of about 330 nm comes predominantly from cells, and is most likely due to the amino acid tryptophan. The intensity of cellular tryptophan-associated autofluorescence is distinguishable in normal, pre-cancerous, and cancerous cells, increasing with an increase in malignancy. The methods use the tryptophan-associated cellular autofluorescence to detect the specifically cellular changes indicative of cancer and early cancer. The methods employ the optical techniques of obtaining either excitation spectra or emission spectra of tissue or cell samples to reveal the change in cellular tryptophan-associated autofluorescence which is indicative of early cancer or cancer. The methods have the clear advantage of involving only a single intensity measurement from a peak in a spectrum, instead of multiple point analysis of a complex waveform. Further, the methods provide rapid optical detection of malignancy.

Cellular tryptophan-associated autofluroescence is not affected by the presence of inflammatory conditions in the same way as it is affected by the presence of malignancy. Inflammation causes a decrease in cellular tryptophan-associated autofluorescence. Therefore, when screening for cancer in patients with inflammatory conditions, a decreased risk exists of obtaining false positive results due to the inflamed tissue in such patients.

In alternative embodiments of the method, whole multiples of the excitation wavelengths are used to obtain the same cellular, tryptophan-associated autofluorescence with multi-photon excitation. The multi-photon excitation approach is especially suitable for penetrating deep tissue, but is also suitable for examining surface tissue.

The methods are applicable to cell or tissue samples from a wide range of organs. For example, the methods are applicable to direct examination of organs such as skin, or by using two way optic fiber probes passed through endoscopes to examine internal organs such as the esophagus, stomach, small intestine, colon, lung, bladder, ureter, cervix, skin, bile duct pancreatic duct, liver, kidney, uterus, ovaries, fallopian tubes, mouth, throat or nasopharynx. Breast tissue and other solid organs are accessible to the method by passing a fiber optic bundle through a needle or trocar. Alternatively, deep tissue or sub-surface measurement can be accomplished using multi-photon excitation or confocal microscopy techniques.

Further, the method is useful for defining a safe margin in real time as a malignancy is being resected by a surgeon, thus avoiding the need for frozen sections to be examined by a pathologist during the surgery as is typically done. The methods are also applicable to automated cell measurements, or cytometry, wherein cell samples of normal or suspected malignant tissue, obtained via tissue brushings, smears or fluid aspirations, are examined, fixed or unfixed, in an automated cytometer for the tryptophan-associated autofluorescence. Thus, any tissue sample being used to practice the method can be a cytological sample obtained through such cytological sampling methods.

Still further, the methods are suitable for use in combination with the use of known dyes, stains and contrast agents because the cellular, tryptophan-associated autofluorescence peak remains unaffected by such agents. These agents include, for example, methylene blue and other dyes or stains commonly used by physicians as contrast agents during, for example, endoscopy procedures or the like. The contrast agents help locate probable areas of pathology. In addition, the methods described herein are consistent with the use of exogenous dyes and fluorescent agents, which also do not affect the tryptophan-associated autofluorescence peak. Therefore, alternative embodiments of the method include the step of applying a suitable contrast agent to a sample of tissue or tissue site to be examined, and then using the contrast agent to identify likely areas of pathology which are then further examined for tryptophan-associated autofluorescence.

Even further, a charge-coupled device can be used in combination with the methods to construct visual images of tissue being examined, wherein the intensity measurements are calibrated to, for example, a color coded scale and displayed on a video monitor. Such an application of the methods allows simultaneous scanning of large areas of tissue.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

This example illustrates the wavelength maxima for cellular autofluorescence peaks corresponding to tryptophan emission.

All fluorescence scans in this an subsequent examples were performed using a spectrofluorometer from Shimatzu Inc., Columbia, Md., with a Xenon lamp and two spectrometers. Emission scans were performed with excitation from 230-350 nm, at 10 nm intervals. The autofluorescence intensity was measured in arbitrary units at 1 nm increments, from 10 nm above the excitation wavelength to 10 less than twice the excitation wavelength. The excitation scans were performed with emission at 350 nm and 400 nm, with excitation from 220 nm to 10 nm less than the emission wavelength.

The autofluorescence of whole tissue samples including samples of normal, dysplastic and malignant colonic tissue were studied. The tissue samples included hyperplastic and adenomatous polyps of the colon, and paired tissue samples from colon, each pair including a sample of normal mucosa and a sample of adenocarcinoma or polyp from the same colon. All tissue samples were immediately frozen in liquid Nitrogen and stored at −70° C. after harvesting. Just prior to spectroscopy, the tissue sample was thawed over ice at room temperature and moistened with phosphate buffered saline (PBS) at a pH of 7.4. Solid tissue of similar shape and size was mounted on a specially constructed sample holder having a black matte surface and placed inside the spectrofluorometer. Spectra from the tissue samples were digitally recorded and later compared to spectra obtained from cells.

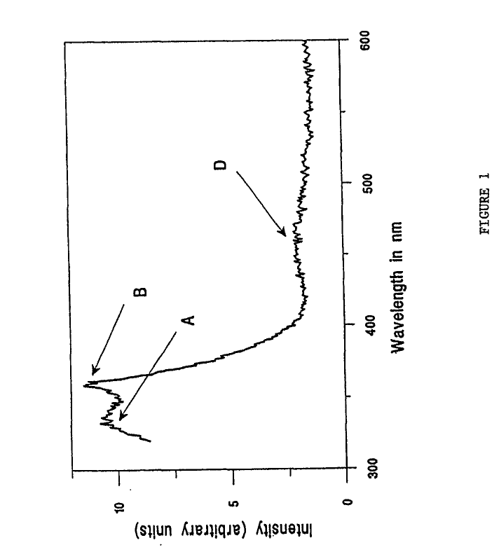

Emission spectra obtained from adenocarcinoma, polyps of the colon (both hyperplastic and adenomatous), and normal colon tissue samples revealed four major emission peaks or maxima, one at about 330 nm (A), one at about 365 nm (B), one at about 385 nm (C), and one at about 450 nm (D). FIG. 1 shows an exemplary emission spectrum, from an adenomatous polyp with excitation at 310 nm. Three of the four major emission peaks A-D were observed, at 330 nm (A), 365 nm B), and 450 nm (D). Different emission peaks appeared as the excitation wavelength was varied, but the four major emission peaks A-D were observed across the range of excitation wavelengths studied. Esophageal, gastric and small intestinal tissue gave similar results. Peak A will be discussed in more detail below. As also will be discussed in more detail below, at least the relatively broad peak D at about 450 nm is likely to be of extracellular origin and therefore not indicative of the cell-specific changes associated with the development of cancer from normal tissue.

Table 1 summarizes the distribution of the major emission maxima A-D for normal (n), adenomatous (a) and cancerous (t) colonic tissue. Table 1 lists the mean wavelength, with standard error (SE), and the range of wavelengths at which each maximum occurred in each tissue type. A one way analysis of variance (ANOVA) was performed on the wavelength distribution of each maximum for each tissue type (e.g. Aₙ, Aₐ and Aₜ), giving the P values as listed in Table 1. For normal tissue samples, N=20; for adenomatous polyps, N=20; for malignant tissue, N=20.

TABLE 1

| | Tissue | Mean | | P | Range |
|---|---|---|---|---|---|
| Maxima | type | Wavelength | SE | value | (nm) |
| A | n | 331.7 | 0.5 | 0.857 | 328-336 |
| | a | 331.8 | 0.5 | | 329-336 |
| | t | 331.4 | 0.6 | | 326-336 |
| B | n | 365.9 | 0.6 | 0.303 | 360-370 |
| | a | 365.1 | 0.6 | | 360-370 |
| | t | 364.7 | 0.5 | | 361-368 |
| C | n | 385.4 | 1.0 | 0.463 | 380-391 |
| | a | 385.3 | 0.8 | | 380-390 |
| | t | 386.6 | 0.6 | | 384-392 |
| D | n | 454.5 | 1.5 | 0.472 | 442-460 |
| | a | 452.1 | 1.4 | | 443-463 |
| | t | 452.9 | 1.3 | | 448-463 |

### EXAMPLE 2

This example illustrates cell specific autofluorescence in cultured cells.

Cultured cells were grown and used to study cell-specific autofluorescence from tissue autofluorescence which includes several nonspecific, extracellular sources such as, for example, collagen and elastin among others. Cultured cells do not contain any extracellular matrix. Cells derived from human colon adenocarcinoma (HT29-18N2) were grown on glass coverslips, in single and multi-layers until seen to be confluent. Cells were washed in PBS prior to spectroscopy to remove growth media.

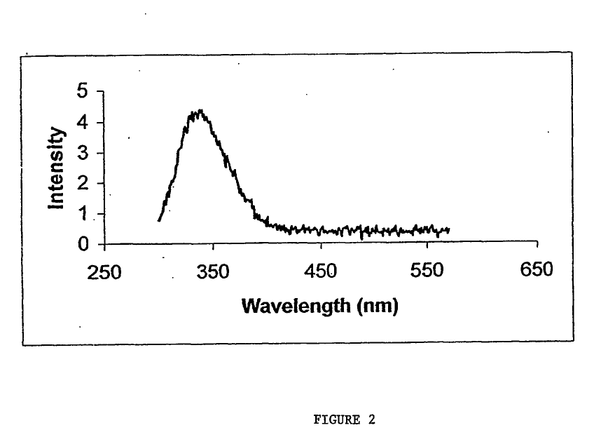

FIG. 2 shows the emission spectrum of a monolayer of human colon adenocarcinoma (HT29-18N2) cells. Excitation of the cultured cells from 280 nm to 330 nm revealed only one major peak S at about 330 nm. Despite excitation at numerous wavelengths, S was the only major peak observed across the range from 280 nm to 700 nm. Thus S, a cellular autofluorescence peak, coincided with peak A, a major tissue autofluorescence peak as shown in FIG. 1. A similar peak at about 330 nm was observed in another cell culture derived from human breast tissue (MCF7). No peaks were observed in cells to match peaks B-D as described in Example 1 and FIG. 1 above.

To see if any other emission maxima might be present under different excitation wavelengths, excitation scans measuring light absorption at different wavelengths were performed. The excitation scans revealed maxima, or strongest absorption of light at 240 nm and 290 nm. Excitation of the cells from 235 nm to 270 nm revealed the S peak at about 330 nm, and an ill defined emission at 260 nm. However, because such low excitation wavelengths are potentially harmful, the 260 nm emission was not studied further. Excitation of the cells at the higher wavelength of 290 nm again revealed only the S peak.

All cultured cells, as well as cells separated from normal and malignant solid tissue including human colon, esophagus and stomach, which were studied at an excitation of 290 nm showed the S peak. No other emission peaks were seen. Previously, it has been proposed that the broad D peak observed in tissue spectra at about 450 nm is due to the molecule NADH which is present in cells. However, none of the autofluorescence spectra from the cultured or extracted cells studied showed an emission peak matching D, thus indicating that the D fluorescence peak is not due to a cellular source.

### EXAMPLE 3

This example illustrates the emission and excitaton spectra of tryptophan which can account for the S peak observed in cells.

The emission and excitation spectra of several known fluorophores were investigated at an excitation wavelength of 290 nm. The fluorophores included Phenylalanine, tryptophan, tyrosine, collagen Type IV, elastin, NADH, and FAD. Only the spectrum of tryptophan, in aqueous solution, produced a peak matching the cellular autofluorescence S peak at about 330 nm, and the tissue autofluorescence peak A at about 330 nm.

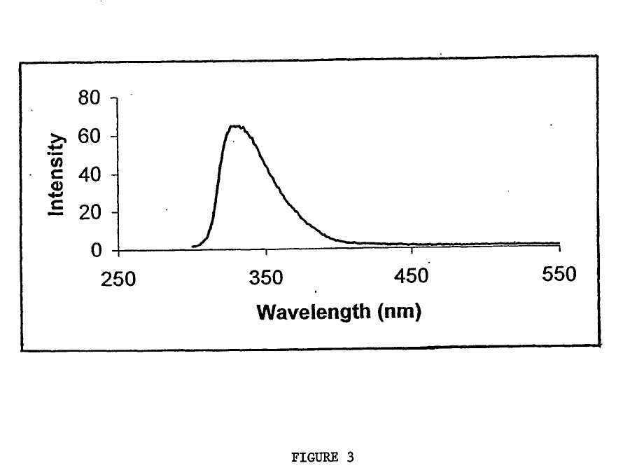

FIG. 3 shows the emission spectrum of tryptophan in aqueous solution, at an excitation wavelength of 290 nm. Thus, tryptophan is highly likely to be the predominant source of the cellular autofluroescence S peak and matching tissue auto fluorescence A peak.

Further, the emission spectrum of NADH (not shown) showed a peak at 460 nm, not 450 nm. These results further support the idea that the broad D peak observed in tissue spectra at about 450 nm is not likely to be due to NADH. Thus, known methods which use the broad D peak to detect cancer are based on the likely false assumption that the D peak is correlated with the cellular marker NADH. Instead, the D peak is likely to be of extracellular origin and thus non-specific to the cellular changes associated with cancer. Further, the magnitude of peak D appears to fall with malignancy, probably due to a higher ratio of cells to extracellular tissue in cancer.

### EXAMPLE 4

This example demonstrates that that increased autofluorescence at about 330 nm in cancerous cells is due to intracellular changes and not explained by greater cell density in cancerous tissue.

Cells were separated from the extracellular matrix of normal and malignant tissue from colon and other organs. Cells separated from tissue and suspended in non-fluorescent solution were confirmed by light microscopy and then placed in a quartz cuvette for spectroscopy. A portion of each cell sample was stained with Trypan Blue and the number of viable cells and total number of cells (cells per cubic millimeter) estimated using known microscopic techniques. The intensity of autofluorescence emitted from the samples was measured at 330 nm, with an excitation wavelength of 290 nm. The fluorescence intensity in each sample at 330 nm was divided by the estimated number of cells. The results from normal and malignant cells were then compared.

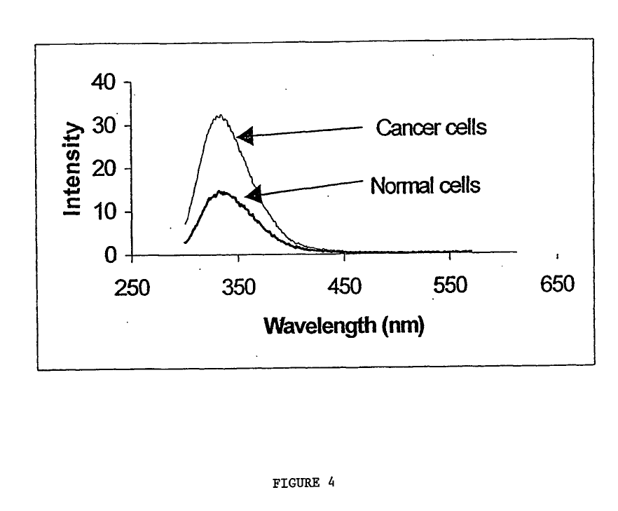

FIG. 4 shows the emission spectra of cells obtained from normal colonic mucosa (normal cells) and cells from adenocarcinoma of the colon (cancer cells). The spectra show the S peak for both cell types at about 330 nm. No other emission peaks were observed, and the spectra were identical to those obtained with the same excitation wavelength from cultured cells of the same type. FIG. 4 also shows that the intensity of the autofluorescence represented by the peak at about 330 nm in spectra of cancerous cells was substantially higher than that observed in the spectra of normal cells.

Table 2 shows the mean autofluorescence in arbitrary units per cell in normal and malignant cells extracted from colon and esophagus. Table 2 shows that the mean autofluorescence per cell was greater in malignant cells than in normal cells.

TABLE 2

Mean autofluorescence
per cell/mm³

| Tissue | Colon | Esophagus |
|---|---|---|
| Normal | 4.5 | 4.9 |
| Malignant | 13.4 | 18.2 |
| Ratio: malignant/normal | 3 | 3.7 |

### EXAMPLE 5

This example demonstrates that fixed cells show autofluorescence which can be used to detect neoplasia.

Cultured human colon adenocarcinoma cells (HT29-18N2) were grown on a coverslip and then fixed. Cells were then kept at room temperature in a closed box. Cells were fixed in a solution of 10% formalin. Emission spectra were obtained at several post-fixation time points, at an excitation wavelength of 290 nm, and the peak autofluorescence intensity at about 330 nm was measured. Spectroscopy was performed at the following times after fixation: 50 minutes, 1 day, 8 days, 14 days and 75 days.

Table 3 shows the peak intensity given in arbitrary units at an emission wavelength of about 330 nm of the cultured cells, measured at the different time periods after fixation. The results show cellular autofluroescence, and more specifically, the cellular tryptophan-associated peak at about 330 nm is maintained for many days at room temperature after cells have been fixed in a standard fixative.

TABLE 3

| Time | Intensity |
|---|---|
| 0 (baseline—no | 5.3 |
| formalin) | |
| 50 min | 4.5 |
| 1 day | 6.4 |
| 8 days | 7.0 |
| 14 days | 5.8 |
| 75 days | 4.9 |

A similar experiment was performed on cells extracted from tissue. The cells were also fixed in a 10% formalin solution and spectroscopy performed as described above. Although the absolute intensities of the cellular typtophan-associated peak was changed, the difference in intensity between normal and malignant cells was maintained. Before fixation, the intensity of malignant cells at about 330 nm was 71% greater than that of normal cells. After fixation, the intensity of the same malignant cells was 125% greater than that of normal cells. Thus, it appears that fixation on formalin not only preserves the tryptophan-associated autofluorescence, but for cell samples amplifies the difference in the autofluorescence intensity between normal and malignant cells. This suggests that the tryptophan-associated autofluorescence can be used for automated cytology. For example, cell smears obtained from organs can be fixed in formalin and transported at room temperature to a facility where cellular autofluorescence is measured and a value for autofluorescence per cell obtained.

### EXAMPLE 6

This example illustrates the cellular source of tryptophan associated with autofluorescence.

Cells were separated from colonic tissue, homogenized and sonicated to rupture the cell wall, and centrifuged to produce a supernatant of cytosol and a membranous sample. The membranous sample was separated and dissolved, and then both the cytosolic supernatant and membranous sample were subjected to spectroscopy. The tryptophan-associated peak at about 330 nm was observed in both fractions, but at much greater intensity in the membranous fraction.

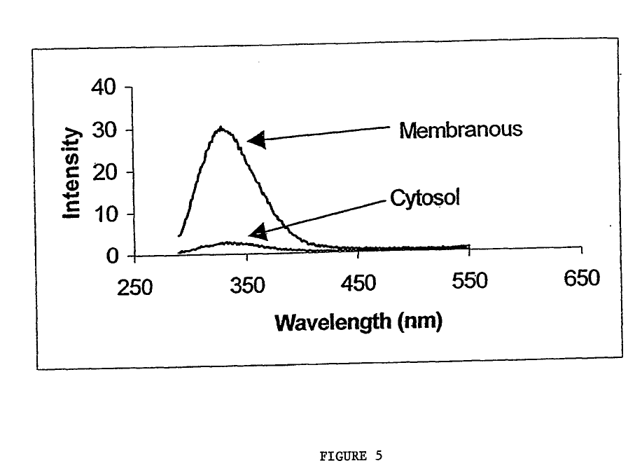

FIG. 5 shows the emission spectrum of membranous and cytosolic fractions derived from cells obtained from normal colonic tissue. The peak observed at about 330 nm is most likely due to tryptophan as discussed in more detail above. Thus it appears that the tryptophan-associated peak originates primarily from a source in the membranous constituents of cells. Such a source is likely to be a membrane-associated protein, group of proteins, or other tryptophan-containing molecules. It is believed that the such a molecule or molecules is present in increased amounts in cancerous and pre-cancerous cells, thus accounting for the increase in intensity of the tryptophan-associated autofluorescence in such cells.

This increase in cellular tryptophan-associated autofluorescence is observable with excitation wavelengths from about 200 nm to about 400 nm. However, excitation with light having a wavelength from about 280 nm to about 300 nm is especially suitable. At excitation wavelengths outside of the range of 280 nm to 300 nm, for example with excitation at 310 nm to 320 nm, other emission peaks appear in addition to the cellular tryptophan-associated peak at about 330 nm, although the peak is still detectable and allows intensity measurements to be made. Even when whole tissue is studied with a suitable excitation wavelength, only autofluorescence from the cells within the tissue is observed. This creates a selective optical window through which cellular autofluorescnce can be observed without interference from extracellular fluorophores. The increase in cellular tryptophan-associated autofluorescence with a peak at about 330 nm, observed with excitation in the wavelength range of about 230 nm to about 350, is thus distinguishable from tissue autofluorescence in malignant tissue.

### EXAMPLE 7

This example illustrates the detection of abnormal cells which constitute metaplasia, hyperplasia, dysplasia or cancer in esophogeal and gastrointestinal tissues.

Tissue samples were obtained from the esophagus, stomach, colon and small intestine. Normal, pre-malignant, i.e. dysplastic, malignant and inflamed tissue samples were obtained and the intensity of the tryptophan-associated peak at about 330 nm measured using an excitation wavelength of 290 nm. A normalized intensity ratio of diseased tissue over normal tissue, was calculated.

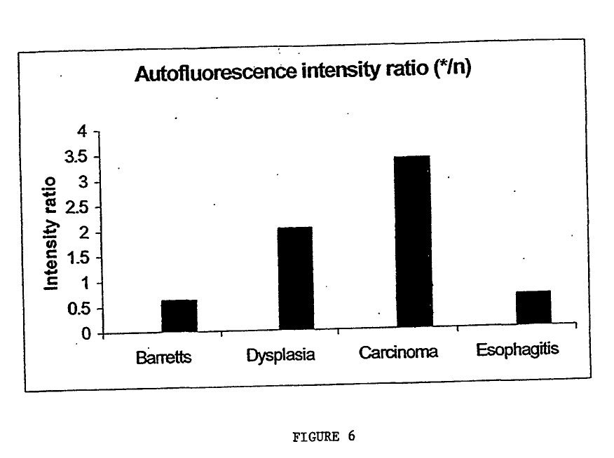

Table 4 shows the mean intensity ratio for esophageal tissue ± the standard error of the mean (SEM). Table 5 shows a repeated of the study using additional tissue samples along with the additional calculation of 95% confidence intervals. FIG. 6 shows the autofluorescence ratio of the tryptophan-associated peak for the different types of esophageal tissue studied. Esophageal metaplasia of squamous cells to columnar cells, the vast majority of which are considered to be Barrett's disease, can potentially develop into malignancy. Esophagitis is an inflammatory condition. Dysplasia, an abnormal state that can progress to malignancy, and carcinoma is an epithelial cell cancer.

The results shown in tables 4 and 5 indicate that autofluorescence at about 330 nm is slightly reduced with inflammation In contrast, the intensity ratio increases for low grade dysplasia and carcinoma. The differences between groups were found to be significant using an analysis of variance. Thus, the single intensity measurement distinguishes inflamed tissue and esophageal metaplasia (with a reduction in the intensity ratio), and dysplastic and malignant tissue (with an increase in the intensity ratio) from normal tissue. This will avoid false positive results during cancer surveillance in patients with inflammatory conditions.

TABLE 4

| | | Mean Intensity |
|---|---|---|
| Tissue | N | Ratio ± SEM |
| Esophageal metaplasia | 17 | 0.62 ± 0.07 |
| Low grade Dysplasia | 8 | 2.01 ± 0.29 |
| Carcinoma | 7 | 3.36 ± 1.03 |
| Esophagitis (inflammation) | 4 | 0.63 ± 0.06 |

The differences between groups were significant: p=0.0002 (ANOVA).

TABLE 5

| | | | 95% |
|---|---|---|---|
| | | Mean Intensity | Confidence |
| Tissue | N | Ratio ± SEM | Intervals |
| Esophageal metaplasia | 10 | 0.63 ± 0.04 | 0.53-0.73 |
| Low Grade Dysplasia | 6 | 1.83 ± 0.26 | 1.17-2.49 |
| Cancer | 7 | 3.18 ± 0.94 | 0.89-5.48 |
| Esophagitis | 5 | 0.76 ± 0.07 | 0.57-0.95 |
| (Inflammation) | | | |

The differences between groups were significant: p=0.0027 (ANOVA).

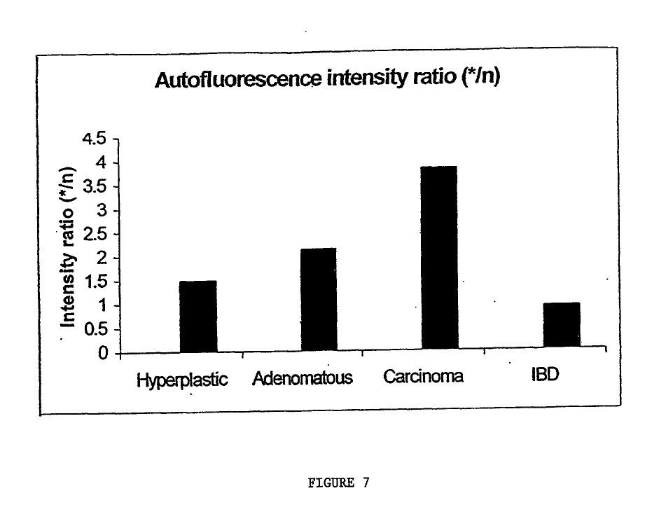

Table 6 shows the mean intensity ratio for colonic tissue ± the standard error of the mean (SEM). Table 7 shows a repeated of the study using additional tissue samples along with the additional calculation of 95% confidence intervals. FIG. 7 shows the autofluorescence ratio of the tryptophan-associated peak for the different types of colonic tissue studied. Hyperplastic polyps are growths lacking malignant potential. They are polyploid, but contain normal cells. Adenomatous polyps are benign growths with malignant potential and include cells which are “a typical”. If detected, adenomatous polyps should be removed, but they are not cancerous. However, if left unremoved, adenomatous polyps can develop into cancer. Inflammatory Bowel Disease (IBD), including Ulcerative Colitis and Crohn's Disease, are chronic inflammatory conditions with an increased risk of cancer.

Referring to Tables 6 and 7, the results indicate that inflammation (IBD) does not enhance, but instead slightly reduces cellular autofluorescence intensity at about 330 nm, similar to the results obtained with inflamed esophageal tissue as described above. In contrast, the intensity ratio is higher for hyperplastic tissue than for normal mucosa. The ratio increases stepwise for adenomas and cancer. The differences between groups were found to be significant using an analysis of variance. Thus, the single intensity measurement distinguishes inflamed colonic tissue (with a reduction in the intensity ratio), and hyperplastic, dysplastic and malignant tissue (with an increase in the intensity ratio) from normal tissue. This will avoid false positive results during cancer surveillance in patients with IBD.

TABLE 6

| | | Mean Intensity |
|---|---|---|
| Tissue | N | Ratio ± SEM |
| Hyperplastic Polyps | 9 | 1.48 ± 0.16 |
| Adenomatous Polyps | 22 | 2.13 ± 0.16 |
| Carcinoma | 11 | 3.81 ± 0.71 |
| IBD | 15 | 0.9 ± 0.04 |

The differences between groups were significant: p=0.001 (ANOVA).

TABLE 7

| | | Mean Intensity | 95% Confidence |
|---|---|---|---|
| Tissue | N | Ratio ± SEM | Intervals |
| Hyperplastic Polyps | 8 | 1.44 ± 0.12 | 1.15-1.72 |
| Adenomatous Polyps | 15 | 1.88 ± 0.12 | 1.63-2.13 |
| Cancer | 10 | 3.08 ± 0.55 | 1.84-4.33 |
| IBD | 9 | 0.82 ± 0.07 | 0.67-0.97 |

The differences between groups were significant: p<0.0001 (ANOVA).

### EXAMPLE 8

This example illustrates the use of excitation scans to detect cancer.

In excitation scans, as opposed to the emission scans described above, the emission wavelength is kept constant and the excitation wavelength varied. The excitation scans for tryptophan, cultured cells, and cells extracted from tissue all reveal a major excitation peak at 290 nm. This peak is also observed in whole tissue, and reveals the presence of cancer in a manner similar to that using emission scans as described above.

Specifically, excitation spectra of normal, dysplastic and cancerous and esophageal tissue were obtained by varying the excitation wavelength from 220 nm to 340 nm. A single intensity measurement of the major tryptophan-associated excitation peak was taken at 290 nm for each tissue type. The mean intensity measurement for each tissue type was normalized to the intensity measurement for normal tissue at 290 nm (mean intensity=1). The ratios of mean emission intensities were: 1.42±0.35 (SE) for low grade dysplasia of esophageal metaplasia (Barrett's disease), (N=6); and 4.03±1.17 for adenocarcinoma (N 9). Thus, the results indicate that single intensity measurements of cellular tryptophan-associated excitation spectra distinguishes cancerous tissue from dysplastic and normal tissue.

All references cited in this specification are hereby incorporated by reference. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results attained.

As various changes could be made in the above methods and apparatus without departing from the scope of the invention, it is intended that all matter contained in the above description and shown in the accompanying drawings shall be interpreted as illustrative and not in a limiting sense.

What is claimed is:

1. A method of detecting neoplasia, the method comprising exposing cells suspected of constituting neoplasia to ultraviolet light; measuring autofluorescence at a wavelength indicative of tryptophan emission; and determining that the cells constitute neoplasia if ratio of emission intensity of the cells to emission intensity of cells which do not constitute neoplasia exceeds about 1.3.

2. The method according to claim 1 wherein measuring autofluorescence at a wavelength indicative of a tryptophan emission comprises measuring autofluorescence at a wavelength which lies within a range of from about 300 nm to about 400 nm.

3. The method according to claim 2 wherein measuring autofluorescence at a wavelength which lies within a range of from about 300 nm to about 400 nm comprises measuring autofluorescence at a wavelength of about 330 nm.

4. The method according to claim 1 wherein the method detects degree of neoplasia and determining that the cells constitute neoplasia if ratio of emission intensity of the cells to emission intensity of cells which do not constitute neoplasia exceeds about 1.3 comprises determining that the cells constitute a more severe degree of neoplasia if the cells show a higher ratio above 1.3 for emission intensity to emission intensity of cells which do not constitute neoplasia.

5. The method according to claim 4 wherein the method detects hyperplasia and determining that the cells constitute a more severe degree of neoplasia if the cells show a higher ratio above 1.3 for emission intensity to emission intensity of cells which do not constitute neoplasia comprises determining that the cells constitute hyperplasia if ratio of emission intensity of the cells to emission intensity of cells which do not constitute neoplasia is higher than about 1.3 but less than about 1.7.

6. The method according to claim 5 wherein the method detects dysplasia and determining that the cells constitute a more severe degree of neoplasia if the cells show a higher ratio above 1.3 for emission intensity to emission intensity of cells which do not constitute neoplasia comprises determining that the cells constitute dysplasia if ratio of emission intensity of the cells to emission intensity of cells which do not constitute neoplasia is higher than about 1.7 but less than about 2.5.

7. The method according to claim 4 wherein the method detects cancerous cells and determining that the cells constitute a more severe degree of neoplasia if the cells show a higher ratio above 1.3 for emission intensity to emission intensity of cells which do not constitute neoplasia comprises determining that the cells constitute cancerous cells if ratio of emission intensity of the cells to emission intensity of cells which do not constitute neoplasia is higher than about 2.5.

8. The method according to claim 2 wherein measuring autofluorescence at a wavelength which lies within a range of from about 300 nm to about 400 nm comprises measuring autofluorescence over a bandwidth which lies within a range of from about 300 nm to about 400 nm.

9. The method according to claim 8 wherein the bandwidth comprises about 10 nm bandwidth.

10. The method according to claim 1 wherein exposing cells suspected of constituting neoplasia to ultraviolet light comprises exposing cells suspected of constituting neoplasia to ultraviolet light comprising a wavelength of 290 nm.

11. The method according to claim 1 wherein the cells are in vivo.

12. The method according to claim 11 wherein exposing cells suspected of constituting neoplasia to ultraviolet light comprises exposing cells suspected of constituting neoplasia to a beam of ultraviolet light delivered directly or by using a lens or mirror or through a fiber optic bundle inserted through a biopsy channel of an endoscope or through a needle.

13. The method according to claim 1 wherein the cells are in vitro.

14. The method according to claim 13 wherein the cells are obtained from a cell washing or a cell brushing.

15. The method according to claim 13 wherein the cells are obtained from a tissue biopsy.

16. The method according to claim 1 wherein the cells which are suspected of constituting a neoplasia are from the same organ type as the cells which do not constitute neoplasia.

17. The method according to claim 1 wherein the cells suspected of constituting a neoplasia are from a gastrointestinal organ, lung, bladder, ureter, cervix, skin, bile duct pancreatic duct, liver, kidney, uterus, ovaries, fallopian tubes, mouth, throat or nasopharynx.

18. The method according to claim 17 wherein the cells suspected of constituting a neoplasia are from the esophagus, stomach, small intestine or colon.

19. The method according to claim 1 wherein the cells are fixed cells.

20. The method according to claim 19 wherein the cells are formalin fixed cells.

21. A method of detecting neoplasia, the method comprising exposing cells suspected of constituting neoplastia to ultraviolet light; measuring autofluorescence emission from the cells over a bandwidth other than a bandwidth of 20 nm wherein the bandwidth lies within a range of from about 300 nm to about 400 nm; and determining that the cells constitute neoplasia if emission is greater than emission of cells which do not constitute neoplasia.

22. The method according to claim 21 wherein determining that the cells constitute neoplasia if emission greater than emission of cells which do not constitute neoplasia comprises determining that the cells constitute neoplasia if ratio of emission intensity of the cells to emission intensity of cells which do not constitute neoplasia exceeds about 1.3.

23. The method according to claim 22 wherein the method detects degree of neoplasia and determining that the cells constitute neoplasia if ratio of emission intensity of the cells to emission intensity of cells which do not constitute neoplasia exceeds about 1.3 comprises determining that the cells constitute a more severe degree of neoplasia if the cells show a higher ratio above 1.3 for emission intensity to emission intensity of cells which do not constitute neoplasia.

24. The method according to claim 23 wherein the method detects hyperplasia and determining that the cells constitute a more severe degree of neoplasia if the cells show a higher ratio above 1.3 for emission intensity to emission intensity of cells which do not constitute neoplasia comprises determining that the cells constitute hyperplasia if ratio of emission intensity of the cells to emission intensity of cells which do not constitute neoplasia is higher than about 1.3 but less than about 1.7.

25. The method according to claim 23 wherein the method detects dysplasia and determining that the cells constitute a more severe degree of neoplasia if the cells show a higher ratio above 1.3 for emission intensity to emission intensity of cells which do not constitute neoplasia comprises determining that the cells constitute dysplasia if ratio of emission intensity of the cells to emission intensity of cells which do not constitute neoplasia is higher than about 1.7 but less than about 2.5.

26. The method according to claim 23 wherein the method detects cancerous cells and determining that the cells constitute a more severe degree of neoplasia if the cells show a higher ratio above 1.3 for emission intensity to emission intensity of cells which do not constitute neoplasia comprises determining that the cells constitute cancerous cells if ratio of emission intensity of the cells to emission intensity of cells which do not constitute neoplasia is higher than about 2.5.

27. The method according to claim 21 wherein the bandwidth comprises about 10 nm bandwidth.

28. The method according to claim 21 wherein exposing cells suspected of constituting neoplasia to ultraviolet light comprises exposing cells suspected of constituting neoplasia to ultraviolet light comprising a wavelength of 290 nm.

29. The method according to claim 21 wherein the cells are in vivo.

30. The method according to claim 29 wherein exposing cells suspected of constituting neoplasia to ultraviolet light comprises exposing cells suspected of constituting neoplasia to a beam of ultraviolet light delivered directly, or by using a lens or mirror or through a fiber optic bundle inserted through a biopsy channel of an endoscope or through a needle.

31. The method according to claim 21 wherein the cells are in vitro.

32. The method according to claim 31 wherein the cells are obtained from a cell washing or a cell brushing.

33. The method according to claim 31 wherein the cells are obtained from a tissue biopsy.

34. The method according to claim 21 wherein the cells which are suspected of constituting a neoplasia are from the same organ type as the cells which do not constitute neoplasia.

35. The method according to claim 21 wherein the cells suspected of constituting a neoplasia are from a gastrointestinal organ, lung, bladder, ureter, cervix, skin, bile duct pancreatic duct, liver, kidney, uterus, ovaries, fallopian tubes, mouth, throat or nasopharynx.

36. The method according to claim 35 wherein the cells suspected of constituting a neoplasia are from the esophagus, stomach, small intestine or colon.

37. The method according to claim 21 wherein the cells are fixed cells.

38. The method according to claim 36 wherein the cells are formalin fixed cells.

39. A method of detecting esophageal metaplasia of squamous cells to columnar cells, the method comprising exposing cells suspected of constituting esophageal metaplasia to ultraviolet light; measuring autofluorescence emission from the cells at a wavelength indicative of a tryptophan emission; and determining that the cells constitute esophageal metaplasia if ratio of emission intensity of the cells to emission intensity of cells which do not constitute esophageal metaplasia is less than about 0.65

40. The method according to claim 39 wherein measuring autofluorescence emission from the cells at a wavelength indicative of a tryptophan emission comprises measuring autofluorescence at a wavelength which lies within about 300 nm to about 400 nm.

41. The method according to claim 40 wherein measuring autofluorescence at a wavelength which lies within about 300 to about 400 nm comprises measuring autofluorescence at 330 nm.

42. The method according to claim 40 wherein measuring autofluorescence at a wavelength which lies within about 300 to about 400 nm comprises measuring autofluorescence emission from the cells over a bandwidth which lies within about 300 nm to about 400 nm.

43. The method according to claim 42 wherein the bandwidth comprises about 10 nm bandwidth.

44. The method according to claim 39 wherein exposing cells suspected of constituting esophageal metaplasia to ultraviolet light comprises exposing cells suspected of constituting esophageal metaplasia to ultraviolet light comprising a wavelength of 290 nm.

45. The method according to claim 39 wherein the cells are in vivo.

46. The method according to claim 45 wherein exposing cells suspected of constituting esopohageal metaplasia to ultraviolet light comprises exposing cells suspected of constituting esophageal metaplasia to a beam of ultraviolet light delivered through a fiber optic bundle inserted through a biopsy channel of an endoscope.

47. The method according to claim 39 wherein the cells are in vitro.

48. The method according to claim 47 wherein the cells are obtained from a cell washing or a cell brushing.

49. The method according to claim 47 wherein the cells are obtained from a tissue biopsy.

50. The method according to claim 39 wherein the cells which do not constitute esophageal metaplasia are esophageal squamous cells.

51. The method according to claim 39 wherein the cells are fixed cells.

52. The method according to claim 51 wherein the cells are formalin fixed cells.

53. A method of detecting inflammation, the method comprising exposing cells suspected of constituting inflammation to ultraviolet light; measuring autofluorescence emission from the cells at a wavelength indicative of a tryptophan emission; and determining that the cells constitute inflammation if ratio of emission intensity of the cells to emission intensity of cells which do not constitute inflammation is less than about 0.75 and greater than about 0.65

54. The method according to claim 53 wherein measuring autofluorescence emission from the cells at a wavelength indicative of a tryptophan emission comprises measuring autofluorescence at a wavelength which lies within about 300 nm to about 400 nm.

55. The method according to claim 54 wherein measuring autofluorescence at a wavelength which lies within about 300 to about 400 nm comprises measuring autofluorescence at 330 nm.

56. The method according to claim 54 wherein measuring autofluorescence at a wavelength which lies within about 300 to about 400 nm comprises measuring autofluorescence emission from the cells over a bandwidth which lies within about 300 nm to about 400 nm.

57. The method according to claim 56 wherein the bandwidth comprises about 10 nm bandwidth.

58. The method according to claim 53 wherein exposing cells suspected of constituting inflammation to ultraviolet light comprises exposing cells suspected of constituting inflammation to ultraviolet light comprising a wavelength of 290 nm.

59. The method according to claim 53 wherein the cells are in vivo.

60. The method according to claim 59 wherein exposing cells suspected of constituting inflammation to ultraviolet light comprises exposing cells suspected of constituting inflammation to a beam of ultraviolet light delivered through a fiber optic bundle inserted through a biopsy channel of an endoscope.

61. The method according to claim 53 wherein the cells are in vitro.

62. The method according to claim 61 wherein the cells are obtained from a cell washing or a cell brushing.

63. The method according to claim 61 wherein the cells are obtained from a tissue biopsy.

64. The method according to claim 39 wherein the cells which do not constitute inflammation are esophageal squamous cells.

65. The method according to claim 53 wherein the cells are fixed cells.

66. The method according to claim 65 wherein the cells are formalin fixed cells.

67. A method of detecting neoplasia in the presence of inflammation, the method comprising exposing a region suspected of containing cells constituting neoplasia in the presence of inflammation to ultraviolet light; measuring autofluorescence at a wavelength indicative of a tryptophan emission spectrum; and determining that the region contains cells constituting neopolasia if emission is greater than emission of cells which do not constitute neoplasia.

68. The method according to claim 67 wherein the method detects degree of neoplasia and determining that the region contains cells constituting neopolasia if emission is greater than emission of cells which do not constitute neoplasia comprises determining that the region contains cells constituting a more severe degree of neoplasia if the region shows a higher ratio of emission intensity to emission intensity of cells which do not constitute neoplasia.

69. The method according to claim 67 wherein determining that the region contains cells constituting neopolasia if emission intensity is higher than emission intensity of cells which do not constitute neoplasia comprises determining that the region contains cells constituting neoplasia if ratio of emission intensity of the region to emission intensity of cells which do not constitute neoplasia exceeds about 1.3.

70. The method according to claim 69 wherein the method detects hyperplasia and determining that the region contains cells constituting neoplasia if ratio of emission intensity of the region to emission intensity of cells which do not constitute neoplasia exceeds about 1.3 comprises determining that the region contains cells constituting hyperplasia if ratio of emission intensity of the region to emission intensity of cells which do not constitute neoplasia is higher than about 1.3 but less than about 1.7.

71. The method according to claim 69 wherein the method detects dysplasia and determining that the region contains cells constituting neoplasia if ratio of emission intensity of the region to emission intensity of cells which do not constitute neoplasia exceeds about 1.3 comprises determining that the region contains cells constituting dysplasia if ratio of emission intensity of the region to emission intensity of cells which do not constitute neoplasia is higher than about 1.7 but less than about 2.5.

72. The method according to claim 69 wherein the method detects cancerous cells and determining that the region contains cells constituting neoplasia if ratio of emission intensity of the region to emission intensity of cells which do not constitute neoplasia exceeds about 1.3 comprises determining that the region contains cancerous cells if ratio of emission intensity of the region to emission intensity of cells which do not constitute neoplasia is higher than about 2.5.

73. The method according to claim 67 wherein measuring autofluorescence at a wavelength indicative of a tryptophan emission comprises measuring autofluorescence at a wavelength which lies within a range of from about 300 nm to about 400 nm.

74. The method according to claim 73 wherein measuring autofluorescence at a wavelength which lies within a range of from about 300 nm to about 400 nm comprises measuring autofluorescence at a wavelength of about 330 nm.

75. The method according to claim 67 wherein measuring autofluorescence at a wavelength which lies within a range of from about 300 nm to about 400 nm comprises measuring autofluorescence over a bandwidth which lies within a range of from about 300 nm to about 400 nm.

76. The method according to claim 75 wherein the bandwidth comprises about 10 nm bandwidth.

77. The method according to claim 67 wherein exposing a region suspected of containing cells constituting neoplasia in the presence of inflammation to ultraviolet light comprises exposing a region suspected of containing cells constituting neoplasia in the presence of inflammation to ultraviolet light comprising a wavelength of 290 nm.

78. The method according to claim 67 wherein the cells are in vivo.

79. The method according to claim 78 wherein exposing cells suspected of constituting neoplasia to ultraviolet light comprises exposing cells suspected of constituting neoplasia to a beam of ultraviolet light delivered directly or by using a lens or mirror or through a fiber optic bundle inserted through a biopsy channel of an endoscope or through a needle.

80. The method according to claim 67 wherein the cells are in vitro.

81. The method according to claim 80 wherein the cells are obtained from a cell washing or a cell brushing.

82. The method according to claim 80 wherein the cells are obtained from a tissue biopsy.

83. The method according to claim 67 wherein the cells which are suspected of constituting a neoplasia are from the same organ type as the cells which do not constitute neoplasia.

84. The method according to claim 67 wherein the cells suspected of constituting a neoplastia are from a gastrointestinal organ, lung, bladder, ureter, cervix, skin, bile duct pancreatic duct, liver, kidney, uterus, ovaries, fallopian tubes, mouth, throat or nasopharynx.

85. The method according to claim 84 wherein the cells suspected of constituting a neoplasia are from the esophagus, stomach, small intestine or colon.

86. The method according to claim 67 wherein the cells are fixed cells.

87. The method according to claim 86 wherein the cells are formalin fixed cells.
